(19) United States
(12) Patent Application Publication
(10) Pub. No.: US 2026/0301936 A1
(43) Pub. Date: Oct. 1, 2026

(54) AUGMENTED UNDERSTANDING THROUGH ANNOTATION OF 3D TELECONFERENCE INTERACTIONS

(71) Applicant: MICROSOFT TECHNOLOGY LICENSING, LLC, Redmond, WA (US)

(72) Inventors: Andréa BRITTO MATTOS LIMA, Sao Paulo (BR); Spencer G FOWERS, Duvall, WA (US); Thiago VALLIN SPINA, Campinas (BR); Deepti Balachandra HEGDE, Baltimore, MD (US)

(21) Appl. No.: 19/093,225

(22) Filed: Mar. 27, 2025

(51) Int. Cl. G16H 40/67; G06F 40/20; G06T 17/20; G06V 10/82; G06V 40/16; G16H 80/00

(52) U.S. Cl. CPC G16H 40/67; G06F 40/20; G06T 17/20; G06V 10/82; G06V 40/174; G16H 80/00

(57) ABSTRACT

Disclosed are techniques for highlighting regions of a subject's body in real-time during a 3D teleconference. In some configurations, images of the subject are captured in real-time and used to generate an input mesh representation of the subject. The input mesh may be displayed to the subject during the teleconference. As a medical practitioner speaks, references to region's of the subject's body are identified. The identified regions may then be highlighted in the display of the input mesh, making it clear what the medical practitioner is referring to. For example, if the practitioner says “I need to further examine the left foot and the right shoulder”, the display of the input mesh may highlight the left foot and the right shoulder of the subject.

### BACKGROUND

Traditional telemedicine systems rely on 2D video conferencing. However, these systems lack the spatial depth and immersive presence required for effective communication in complex medical scenarios. This limitation is particularly significant in plastic and reconstructive surgery where patients often struggle to understand intricate procedures such as tumor removal and flap-based reconstruction. These procedures may involve the transplantation of skin, blood vessels, and other tissues from donor sites on the body. Failing to understand the full extent of a procedure can cause confusion, anxiety, and regret in a patient.

It is with respect to these and other considerations that the disclosure made herein is presented.

### SUMMARY

Disclosed are techniques for highlighting regions of a subject's body in real-time during a 3D teleconference. In some configurations, images of the subject are captured in real-time and used to generate an input mesh representation of the subject. The input mesh may be displayed to the subject during the teleconference. As a medical practitioner speaks, references to region's of the subject's body are identified. The identified regions may then be highlighted in the display of the input mesh, making it clear what the medical practitioner is referring to. For example, if the practitioner says “I need to further examine the left foot and the right shoulder”, the display of the input mesh may highlight the left foot and the right shoulder of the subject.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 illustrates generating an input mesh representation of a subject.

FIG. 2 illustrates using a parameterized model of a subject to generate a model-generated mesh of the subject.

FIG. 3 illustrates highlighting particular regions of a subject in a display of a segmented input mesh representation of the subject.

FIG. 4 illustrates obtaining a list of one or more names of regions of a subject.

FIG. 5 is a flow diagram of an example method for augmented understanding through annotation of 3D teleconference interactions.

FIG. 6 is a computer architecture diagram illustrating an illustrative computer hardware and software architecture for a computing system capable of implementing aspects of the techniques and technologies presented herein.

### DETAILED DESCRIPTION

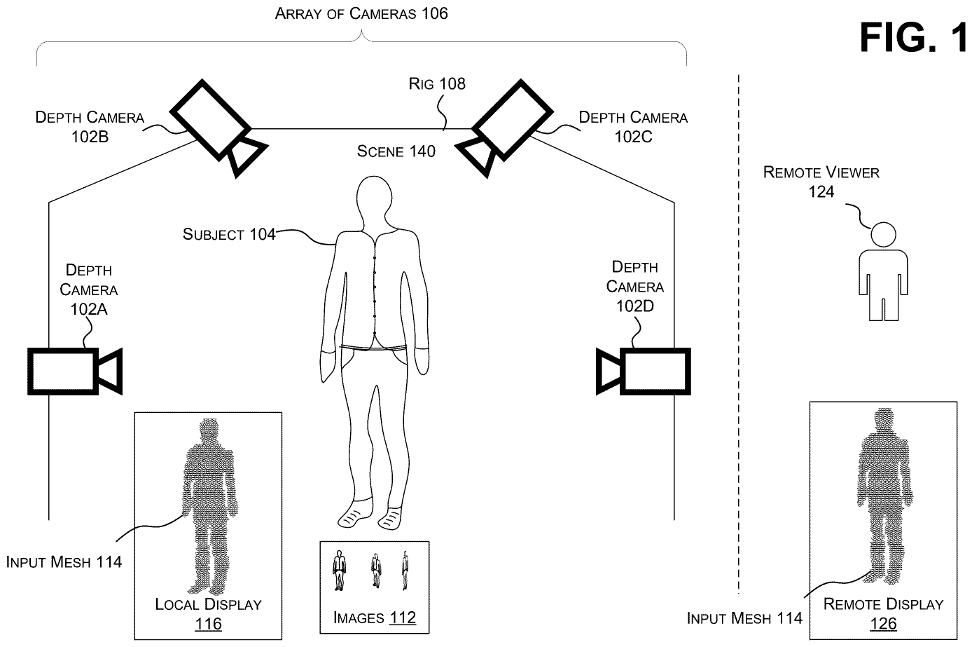

FIG. 1 illustrates generating an input mesh representation of a subject. Multiple depth cameras 102 capture images 112 of subject 104. Depth cameras 102 may be attached to rig 108, which holds depth cameras 102 in place so as to view scene 140 from different perspectives. Depth cameras 102, which are collectively referred to as array of cameras 106, may capture color images of scene 140 as well as depth maps that precisely measure a distance for each pixel. For example, depth camera 102A may include a synchronized RGB camera for capturing color and a depth camera for capturing depth. Additionally, or alternatively depth cameras 102 may not include a dedicated depth sensor. For these cameras, depth information may be inferred from the red, green, and blue values of the RGB camera. Depth information allows for the creation of 3D representations of scene 140 or objects within it.

Depth cameras 102 use various technologies to capture depth information, including: Time-of-Flight (ToF), Structured Light, and Stereo Vision. Time of Flight cameras emit light or infrared signals and measure the time it takes for the signal to bounce back to the camera. By calculating the time delay, the camera can determine the distance between the camera and objects in the scene. Structured light cameras project a pattern of light onto the scene and analyze the distortion of the pattern as it interacts with objects. This distortion is used to calculate depth information based on the known pattern. Stereo cameras use two or more camera lenses to capture the same scene from slightly different angles. By comparing the disparities between the images, the depth information can be computed using triangulation techniques.

Depth camera image data of images 112 is used to generate input mesh 114. Input mesh 114 is a 3-dimensional (3D) representation of subject 104. Input mesh 114 is depicted with triangles, but any polygon, combination of polygons, splines, or other mathematical descriptions may similarly be used to describe the contour of subject 104. Input mesh 114 may be generated in real-time as subject 104 moves about scene 140. Dynamically updating input mesh 114 adds a time dimension, resulting in a four-dimensional (4D) representation of subject 104.

In some scenarios, subject 104 is a medical patient and input mesh 114 is generated as part of a telemedicine application. Input mesh 114 may be displayed for a medical practitioner or other remote viewer 124 sitting remotely from subject 104. For example, a medical practitioner may view input mesh representation on remote display 126 while remotely diagnosing, advising, or otherwise interacting with subject 104. Input mesh 114 may also be displayed locally on local display 116.

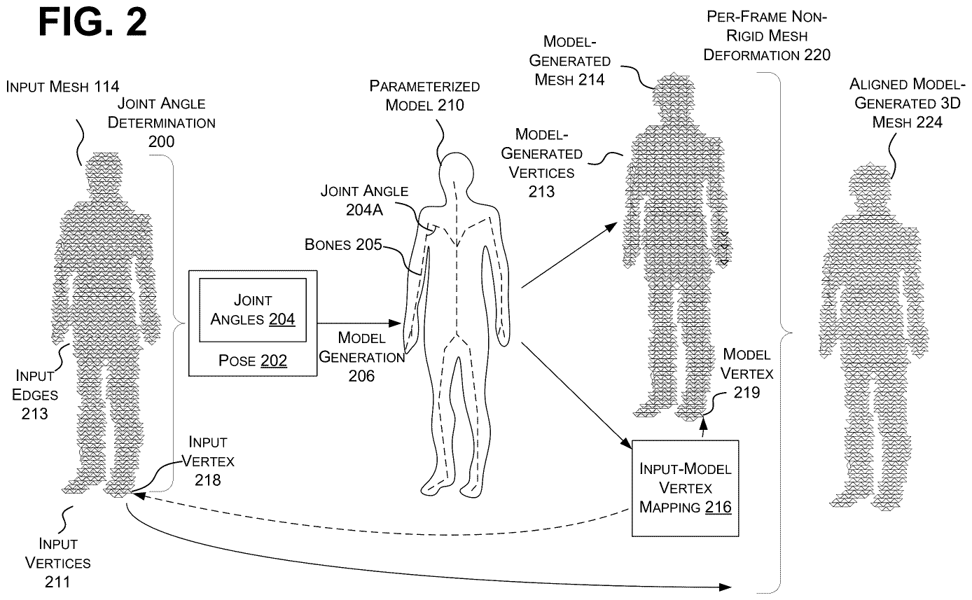

FIG. 2 illustrates using a parameterized model of a subject to generate a model-generated mesh of the subject. Input mesh 114 is depicted as a number of input vertices 211 connected by input edges 213. As discussed above, input vertices 211 and input edges 213 may form triangles, quadrilaterals, or other geometric shapes.

Joint angle determination 200 is performed on input mesh 114, yielding pose 202. Pose 202 describes the locations and orientations of regions of subject 104. In some configurations, joint angles 204 of pose 202 describe the relative positions of different regions, such as the position of a forearm in relation to the hand of subject 104. In some configurations, a computer vision framework such as FRANKMOCAP estimates pose 202 using deep learning-based methods. Specifically, deep neural networks trained on large-scale datasets infer articulated 3D pose 202.

Pose 202 may be used for model generation 206 of parameterized model 210. Parameterized model 210 represents human shape and pose, capturing the geometric structure and the articulated motion of the human body. One example of parameterized model 210 is a Skinned Multi-Person Linear (SMPL) model. Computer vision frameworks, such as FRANKMOCAP, may be capable of generating parameterized model 210 directly from input mesh 114.

Parameterized model 210 may itself be built on a model-generated mesh 214 of a human body, consisting of thousands of model-generated vertices 213 that represent the body's surface. Model-generated vertices 213 may be organized to capture detailed anatomical features, such as joints, limbs, and body contours. Model 210 is parametric in that it can be modified by adjusting a small set of control parameters that define the body's shape, pose, and identity.

Shape Parameters represent the overall shape of the body, including factors like height, weight, and body proportions. These parameters allow parametric model 210 to generate different body types, from thin to muscular or broader physiques. Pose Parameters control the body's posture, allowing for the simulation of different poses and joint angles 204. These parameters capture the relative rotations of the body's bones and joints.

Parameterized model 210 may use a technique called skinning to attach input mesh 114 to a skeleton of bones 205. When bones 205 move, the input mesh 114 deforms accordingly to simulate realistic body motion.

Model generation 206 may also generate input-model vertex mapping 216. Input-model vertex mapping 206 maps vertices in input mesh 114 to vertices in model-generated mesh 214. As illustrated, input vertex 218 of input mesh 114 is mapped to corresponding model vertex 219 of model-generate mesh 214. Input-model vertex mapping 216 allows relevant regions of subject 104 that are identified with parameterized model 210 to be identified in input mesh 114.

In some configurations, per-frame non-rigid mesh deformation 220 modifies model-generated mesh 214 to conform to the pose of subject 104. Specifically, model-generated mesh 214, which is based on parameterized model 210, is deformed to conform with input mesh 114, generating aligned model-generated 3D mesh 224. Non-rigid mesh deformation 220 may be performed using an iterative closest point algorithm or learned mappings. Learned mappings refers to applying a machine learning model, such as a multi-layered perceptron, that has been trained to infer the adjustments to model-generated mesh 214 that will conform it to input mesh 114. Other types of machine learning models are similarly contemplated, as are techniques that do not rely on perceptrons or other machine learning techniques.

Model-generated mesh 214 may not already conform with input mesh 114 because model-generated mesh 214 is derived from parameterized model 210, which may not completely represent the nuances of input mesh 114. For example, parameterized model 210 may be defined by 24 joints. While effective for many applications, and while beneficial for reasons of efficiency, a small, limited number of joints may not allow for an acceptable percentage of model-generated vertices 213 to match the contours of input vertices 211.

In some configurations, per-frame non-rigid mesh deformation 220 is accomplished by moving the vertices of model-generated mesh 214 to be aligned with the locations of the corresponding vertices in input mesh 114. For example, model vertex 219, which is associated with input vertex 218, may be moved in the X, Y, and/or Z axis to be in a location relative to the rest of model-generated vertices 213 as vertex 218 is to input vertices 211.

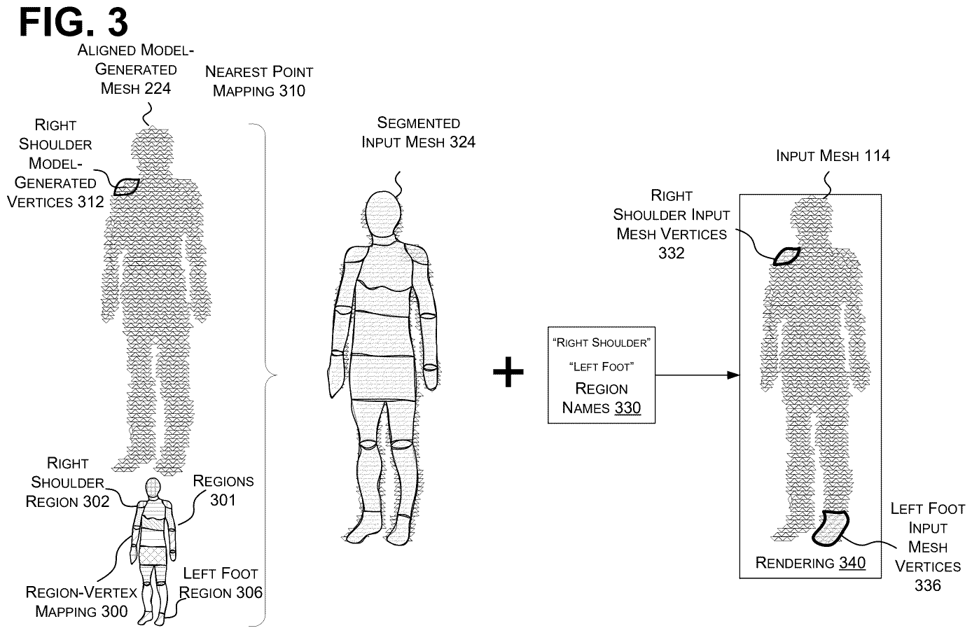

FIG. 3 illustrates highlighting particular regions of a subject in a display of a segmented input mesh representation of the subject. Region-vertex mapping 300 depicts a pre-defined segmentation of parameterized model 210 into regions 301 such as right shoulder region 302 and left foot region 306. Regions 301 may represent any portion of subject 204, including basic segmentation into primary anatomical regions, joint-based regions, and fine-grained anatomical regions. Primary anatomical regions include the head, upper limbs, lower limbs, and torso including the chest, abdomen, and back. Joint-based segmentation regions include the pelvis, spine (upper, mid, and lower), head, and neck, as well as left and right shoulders, elbows, wrists, hips, knees, ankles, and feet.

Parameterized model 210 may apply region-vertex mapping 300 to identify vertices that are associated with a particular region. For example, right shoulder model-generated vertices 312 is a collection of vertices, and adjoining edges, that correspond to right shoulder region 302.

Nearest point mapping 310 applies one or more algorithms to map vertices of aligned model-generated mesh 224 to input mesh 114, yielding segmented input mesh 324. Segmented input mesh 324 labels input vertices 211 according to regions 301, enabling individual regions of input mesh 114 to be efficiently identified, highlighted, or otherwise operated on. In some configurations, regions with different levels of granularity can be considered, allowing finer-grained mapping of input vertices 211 to a larger number of regions 301 or a coarser-grained mapping of input vertices 211 to a smaller number of regions 301. The level of granularity of regions 301 may be set explicitly by subject 104 or remote viewer 124 or another user. Additionally, or alternatively, the level of granularity of regions 301 may be set automatically to correspond to a level of detail of input mesh 114, e.g., the larger the number of input vertices 211 the more regions 301 may be applied.

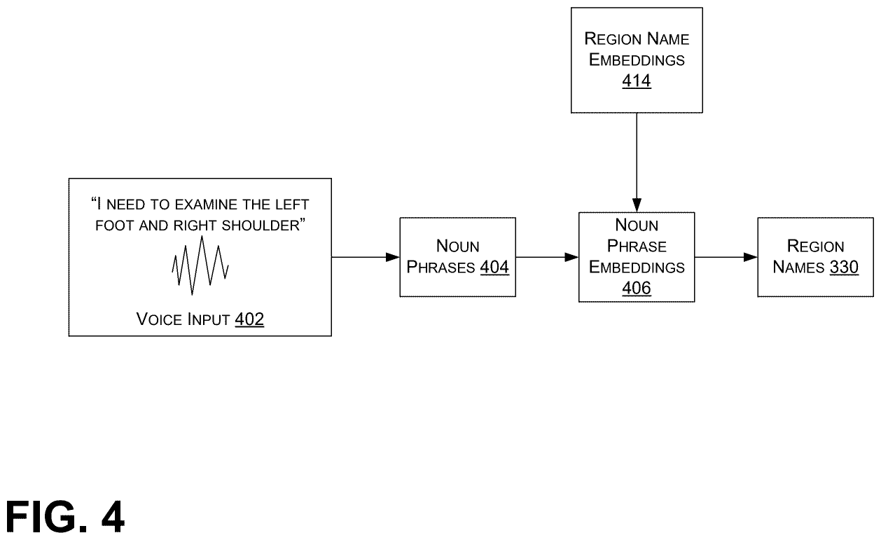

Region names 330 is a collection of one or more names that refer to one or more regions 301 of subject 104. Word embeddings may be used to represent individual region names 330 in a vector space. Names that do not refer to a region 301 of subject 104 are ignored. Region names 330 may be obtained from analyzing text, voice, video, or other media. Additionally, or alternatively, region names 330 may include dynamically identified regions, such as an area of concern identified by a machine learning model. In some configurations region names 330 may be selected from a predefined list of names, while in other configurations region names 330 may be identified dynamically. One example of obtaining region names 330 is discussed below in conjunction with FIG. 4.

Rendering 340 depicts how input mesh 114 can appear in local display 116. As illustrated, region names 330 includes “right shoulder” and “left foot”, both of which exist in a predefined list of regions supported by parameterized model 210. Accordingly, rendering 340 highlights right shoulder input mesh vertices 332. These vertices are obtained by using region-vertex mapping 300 to identify right shoulder model-generated vertices 312. Input-model vertex mapping 216 is then used to identify the corresponding right shoulder input mesh vertices 332 of input mesh 114. A similar process is used to identify and highlight left foot input mesh vertices 336.

Rendering 340 may include speech or text of remote viewer 124, such as an explanation by a medical practitioner of where a skin graft will be taken from. This content may be reproduced by local display 116 while different regions of input mesh 114 are highlighted. However, medical practitioners often use language that is too technical to be understood by the average person. Also, language barriers may interfere with the ability of the remote viewer 124 to effectively communicate with subject 104. To address these issues, in some configurations, text or speech generated by remote viewer 124 is processed by a language simplification and translation engine that adapts explanations to a patient's preferred language and knowledge base. Similarly, a cultural sensitivity engine may adapt explanations based on cultural background.

In some configurations, parameterized model 210, input mesh 114, or other representations of subject 104 may be analyzed in real time to determine that subject 104 is confused by an explanation. Biofeedback may also be monitored to detect a state of confusion in subject 104. When confusion is detected, the language simplification and translation engine may be configured to simplify the explanation further.

FIG. 4 illustrates obtaining a list of one or more names of regions of a subject. FIG. 4 illustrates obtaining a list of region names 330 from voice input 402, which could be a conversation between a medical practitioner and subject 104. As illustrated, voice input 402 has captured someone saying “I need to examine the left foot and right shoulder.” In some configurations, voice input 402 may be analyzed to identify a list of noun phrases 404, such as “I”, “left foot”, and “right shoulder.” Voice input 402 may first be transcribed as text before identifying noun phrases 404.

In some configurations, noun phrases 404 are converted to noun phrase embeddings 406. In this context, an embedding refers to a multidimensional vector that represents the meaning of a noun phrase in an embedding space. Tools such as word2vec may be used to obtain noun phrase embeddings 406.

Noun phrase embeddings 406 may be compared against region name embeddings 414 embeddings that represent a predefined list of regions, and which are obtained from the same embedding space as was used to generate noun phrase embeddings 406. In this way, a noun phrase embedding 406 that is within a defined distance of a region name embedding 414 is considered a match, and is added to region names 330.

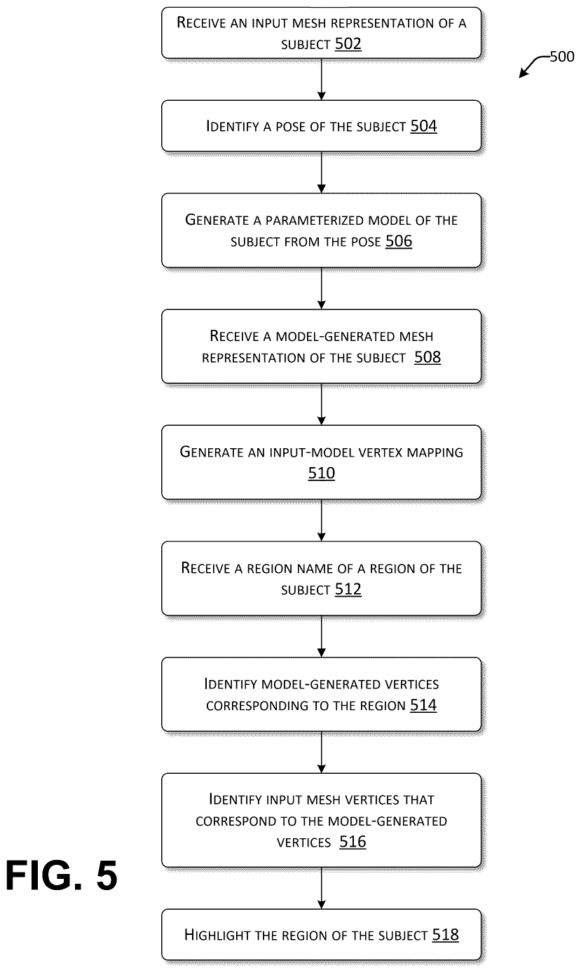

With reference to FIG. 5, routine 500 begins at operation 502, where input mesh 114 is received. In some configurations, input mesh 114 is extracted from images 112 that contain depth and color information of subject 104.

Next at operation 504, pose 202 of subject 104 is identified. Pose 202 may include one or more joint angles 204.

Next at operation 506, pose 202 is used to generate a parameterized model 210 of subject 104. Parameterized model 210 may be parameterized on joint angles 204, indicating the relative positions and orientations of particular regions of subject 104.

Next at operation 508, a model-generated mesh 214 of subject 104 is received. Model-generated mesh 214 may include the same or different numbers and/or types of vertices, edges, triangles, and/or polygons as input mesh 114.

Next at operation 510, input-model vertex mapping 216 is generated. Input-model vertex mapping 216 maps input vertices 211 such as input vertex 218 to model-generated vertices 213 such as model vertex 219. In some configurations, input-model vertex mapping 216 is generated by, for each vertex of input vertices 211, finding a nearest model-generated vertex 213. In some configurations, input-model vertex mapping 216 is generated after aligning and/or deforming model-generated vertices 213 to conform to the shape and location of input vertices 211.

Next at operation 512, a region name 330 of a region 302 is received. The region name 330 may be a noun phrase extracted from a stream of text or voice content, such as during a conversation with a medical practitioner.

Next at operation 514, model-generated vertices 312 that correspond with region 302 are identified. In some configurations, region-vertex mapping 300 is used to determine which model-generated vertices 312 correspond with region 302. In this way, the number and type of regions that can be highlighted is configurable by updating region-vertex mapping 300. For example, adding veins to the list of regions may be accomplished by adding a mapping from veins to vertices to region-vertex mapping 300.

Next at operation 516, input mesh vertices 332 corresponding to the identified model-generated vertices 312 are identified. In some configurations, input-model vertex mapping 216 is used to identify input mesh vertices 332 from model-generated vertices 312 that correspond to region 302.

Next at operation 516, region 302 of subject 104 is highlighted. For example, input mesh vertices 332 corresponding to region 302 may be colored distinctly.

The particular implementation of the technologies disclosed herein is a matter of choice dependent on the performance and other requirements of a computing device. Accordingly, the logical operations described herein are referred to variously as states, operations, structural devices, acts, or modules. These states, operations, structural devices, acts, and modules can be implemented in hardware, software, firmware, in special-purpose digital logic, and any combination thereof. It should be appreciated that more or fewer operations can be performed than shown in the figures and described herein. These operations can also be performed in a different order than those described herein.

It also should be understood that the illustrated methods can end at any time and need not be performed in their entireties. Some or all operations of the methods, and/or substantially equivalent operations, can be performed by execution of computer-readable instructions included on a computer-storage media, as defined below. The term “computer-readable instructions,” and variants thereof, as used in the description and claims, is used expansively herein to include routines, applications, application modules, program modules, programs, components, data structures, algorithms, and the like. Computer-readable instructions can be implemented on various system configurations, including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These operations, structural devices, acts, and modules may be implemented in software, in firmware, in special purpose digital logic, and any combination thereof.

For example, the operations of the routine 500 are described herein as being implemented, at least in part, by modules running the features disclosed herein can be a dynamically linked library (DLL), a statically linked library, functionality produced by an application programing interface (API), a compiled program, an interpreted program, a script or any other executable set of instructions. Data can be stored in a data structure in one or more memory components. Data can be retrieved from the data structure by addressing links or references to the data structure.

Although the following illustration refers to the components of the figures, it should be appreciated that the operations of the routine 500 may be also implemented in many other ways. For example, the routine 500 may be implemented, at least in part, by a processor of another remote computer or a local circuit. In addition, one or more of the operations of the routine 500 may alternatively or additionally be implemented, at least in part, by a chipset working alone or in conjunction with other software modules. In the example described below, one or more modules of a computing system can receive and/or process the data disclosed herein. Any service, circuit or application suitable for providing the techniques disclosed herein can be used in operations described herein.

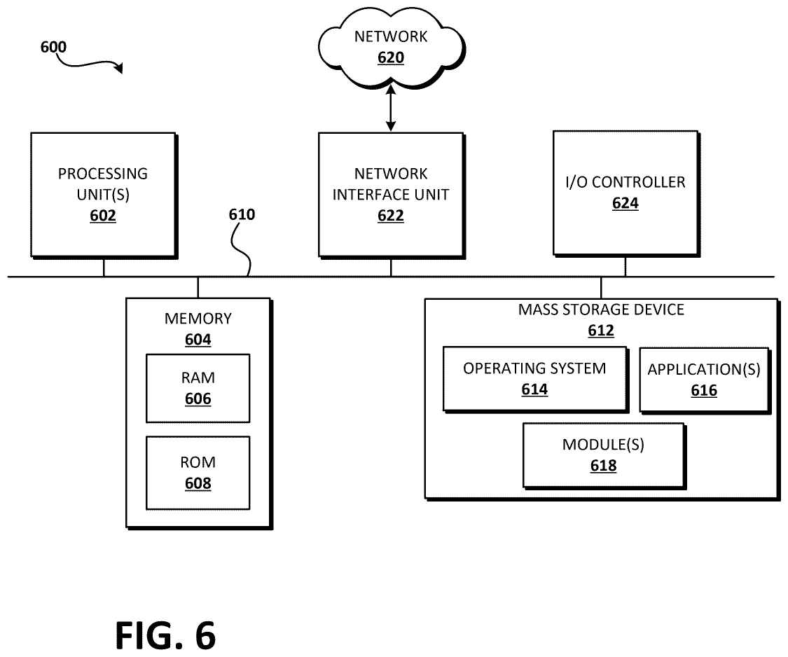

FIG. 6 shows additional details of an example computer architecture 600 for a device, such as a computer or a server configured as part of the systems described herein, capable of executing computer instructions (e.g., a module or a program component described herein). The computer architecture 600 illustrated in FIG. 6 includes processing unit(s) 602, a system memory 604, including a random-access memory 606 (“RAM”) and a read-only memory (“ROM”) 608, and a system bus 610 that couples the memory 604 to the processing unit(s) 602.

Processing unit(s), such as processing unit(s) 602, can represent, for example, a CPU-type processing unit, a GPU-type processing unit, a neural processing unit, a field-programmable gate array (FPGA), another class of digital signal processor (DSP), or other hardware logic components that may, in some instances, be driven by a CPU. For example, and without limitation, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), etc.

A basic input/output system containing the basic routines that help to transfer information between elements within the computer architecture 600, such as during startup, is stored in the ROM 608. The computer architecture 600 further includes a mass storage device 612 for storing an operating system 614, application(s) 616, modules 618, and other data described herein.

The mass storage device 612 is connected to processing unit(s) 602 through a mass storage controller connected to the bus 610. The mass storage device 612 and its associated computer-readable media provide non-volatile storage for the computer architecture 600. Although the description of computer-readable media contained herein refers to a mass storage device, it should be appreciated by those skilled in the art that computer-readable media can be any available computer-readable storage media or communication media that can be accessed by the computer architecture 600.

Computer-readable media can include computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including but not limited to random access memory (RAM), static random-access memory (SRAM), dynamic random-access memory (DRAM), phase change memory (PCM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer storage media does not include communication media. That is, computer-readable storage media does not include communications media consisting solely of a modulated data signal, a carrier wave, or a propagated signal, per se.

According to various configurations, the computer architecture 600 may operate in a networked environment using logical connections to remote computers through the network 620. The computer architecture 600 may connect to the network 620 through a network interface unit 622 connected to the bus 610. The computer architecture 600 also may include an input/output controller 624 for receiving and processing input from a number of other devices, including a keyboard, mouse, touch, or electronic stylus or pen. Similarly, the input/output controller 624 may provide output to a display screen, a printer, or other type of output device.

It should be appreciated that the software components described herein may, when loaded into the processing unit(s) 602 and executed, transform the processing unit(s) 602 and the overall computer architecture 600 from a general-purpose computing system into a special-purpose computing system customized to facilitate the functionality presented herein. The processing unit(s) 602 may be constructed from any number of transistors or other discrete circuit elements, which may individually or collectively assume any number of states. More specifically, the processing unit(s) 602 may operate as a finite-state machine, in response to executable instructions contained within the software modules disclosed herein. These computer-executable instructions may transform the processing unit(s) 602 by specifying how the processing unit(s) 602 transition between states, thereby transforming the transistors or other discrete hardware elements constituting the processing unit(s) 602.

The term “generative model,” as used herein, refers to a machine learning model employed to generate new content. One type of generative model is a “generative language model,” which is a model that can generate new sequences of text given some input. One type of input for a generative language model is a natural language prompt, e.g., a query potentially with some additional context. For instance, a generative language model can be implemented as a neural network, e.g., a long short-term memory-based model, a decoder-based generative language model, etc. Examples of decoder-based generative language models include versions of models such as GPT, BLOOM, PaLM, Mistral, Gemini, and/or LLaMA. Generative language models can be trained to predict tokens in sequences of textual training data. When employed in inference mode, the output of a generative language model can include new sequences of text that the model generates.

Another type of generative model is a “generative image model,” which is a model that generates images or video. For instance, a generative image model can be implemented as a neural network, e.g., a generative image model such as one or more versions of Stable Diffusion, DALL-E, Sora, or GENIE. A generative image model can generate new image or video content using inputs such as a natural language prompt and/or an input image or video. One type of generative image model is a diffusion model, which can add noise to training images and then be trained to remove the added noise to recover the original training images. In inference mode, a diffusion model can generate new images by starting with a noisy image and removing the noise. Note also that generative image models can generate videos, and the term “image” also encompasses two-dimensional and three-dimensional video.

In some cases, a generative model can be multi-modal. For instance, a model may be capable of using various combinations of text, images, video, audio, application states, code, or other modalities as inputs and/or generating combinations of text, images, video, audio, application states, or code or other modalities as outputs. Here, the term “generative language model” encompasses multi-modal generative models where at least one mode of output includes natural language tokens. Likewise, the term “generative image model” encompasses multi-modal generative models where at least one mode of output includes images or video. Examples of multi-modal models include certain GPT variants such as GPT-4o, Gemini, Chameleon, etc. Multi-modal models can also include lightweight models such as Phi-3-Vision-128K-Instruct.

In addition, some generative models can include computer vision capabilities. These models are capable of recognizing objects in input images. The term “computer vision model” encompasses multi-modal models such as one or more versions of CLIP (Contrastive Language-Image Pre-Training) and BLIP (Bootstrapping Language-Image Pre-Training). Note the term “computer vision model” also encompasses non-generative models, such as ResNet, Faster-RCNN, etc. The term “vision language model” refers to any multi-modal generative model that can generate text describing images or videos, including CLIP, BLIP, Vision-and-Language BERT, Flamingo, Chameleon, etc.

The term “prompt,” as used herein, refers to input provided to a generative model that the generative model uses to generate outputs. A prompt can be provided in various modalities, such as text, an image, audio, video, etc. The term “language generation prompt” refers to a prompt to a generative model where the requested output is in the form of natural language. The term “image generation prompt” refers to a prompt to a generative model where the requested output is in the form of an image.

The term “machine learning model” refers to any of a broad range of models that can learn to generate automated user input and/or application output by observing properties of past interactions between users and applications. For instance, a machine learning model could be a neural network, a support vector machine, a decision tree, a clustering algorithm, etc. In some cases, a machine learning model can be trained using labeled training data, a reward function, or other mechanisms, and in other cases, a machine learning model can learn by analyzing data without explicit labels or rewards.

The present disclosure is supplemented by the following example clauses:

Example 1: A method comprising: receiving an input mesh of a subject; identifying a pose of the subject from the input mesh; generating a parameterized model of the subject from the pose; receiving a model-generated mesh of the subject from the parameterized model; generating an input-model vertex mapping from vertices of the model-generated mesh to corresponding vertices of the input mesh; receiving a region name of a region of the subject; identifying a plurality of model-generated vertices of the model-generated mesh corresponding to the region name; identifying, from the input-model vertex mapping, a plurality of input mesh vertices that correspond to the plurality of model-generated vertices; and highlighting the region of the subject by modifying how the plurality of input mesh vertices appear in a rendering of the input mesh of the subject.

Example 2: The method of Example 1, wherein the representation comprises a real-time 3D representation of the subject.

Example 3: The method of Example 1, wherein the subject comprises a medical patient and the region name is identified by a medial professional.

Example 4: The method of Example 1, wherein the pose is defined by joint positions inferred from the input mesh of the subject.

Example 5: The method of Example 4, wherein the parameterized model represents the subject with a plurality of body parts connected by a plurality of joints, and wherein the parameterized model is parameterized with the joint positions and joint angles of the pose.

Example 6: The method of Example 1, further comprising: moving one or more vertices of the model-generated mesh of the subject to align with one or more corresponding vertices of the input mesh of the subject.

Example 7: The method of Example 6, wherein the one or more vertices of the model-generated representation are moved independently of each other.

Example 8: The method of Example 7, wherein the one or more vertices of the model-generated representation are moved according to a multi-layer perceptron trained to conform a pair of mesh representations.

Example 9: A system comprising: a processing unit; and a non-transitory computer-readable storage medium having computer-executable instructions stored thereupon, which, when executed by the processing unit, cause the processing unit to: receive an input mesh of a medical patient; identify a pose of the medical patient from the input mesh; generate a parameterized model of the medical patient from the pose; receive a model-generated mesh of the medical patient from the parameterized model; generate an input-model vertex mapping from vertices of the model-generated mesh to corresponding vertices of the input mesh; receive a region name of a region of the medical patient from a medical practitioner; identify a plurality of model-generated vertices of the model-generated mesh corresponding to the region name; identify, from the input-model vertex mapping, a plurality of input mesh vertices that correspond to the plurality of model-generated vertices; and highlight the region of the medical patient by modifying how the plurality of input mesh vertices appear in a rendering of the input mesh of the subject.

Example 10: The system of Example 9, wherein the input-model generated vertex mapping associates a vertex of the model-generated mesh at a location of the medical patient with a vertex of the input mesh that is closest to the location of the medical patient.

Example 11: The system of Example 9, wherein the region name is identified by extracting a noun phrase from text or speech produced by the medical practitioner, computing an embedding vector of the noun phrase, and determining that the embedding vector is within a defined distance of an embedding vector representation of the region of the medical patient.

Example 12: The system of Example 11, wherein the computer-executable instructions further cause the processing unit to: reproduce the text or speech received from the medical practitioner with a local display that is observable by the medical patient.

Example 13: The system of Example 12, wherein the text or speech received from the medical practitioner is processed by a language simplification engine to make the text or speech more understandable to the medical patient.

Example 14: The system of Example 9, wherein the region is highlighted by changing a color of the plurality of input mesh vertices displayed by a local display.

Example 15: The system of Example 9, wherein the computer-executable instructions further cause the processing unit to: analyze facial expression and body language of the medical patient to detect that the medical patient is confused; and alert the medical practitioner that the medical patient is confused.

Example 16: A non-transitory computer-readable storage medium having encoded thereon computer-readable instructions that when executed by a processing unit causes a system to: receive an input mesh of a subject; identify a pose of the subject from the input mesh; generate a parameterized model of the subject from the pose; receive a model-generated mesh of the subject from the parameterized model; generate an input-model vertex mapping from vertices of the model-generated mesh to corresponding vertices of the input mesh; receive a region name of a region of the subject; identify a plurality of model-generated vertices of the model-generated mesh corresponding to the region name; identify, from the input-model vertex mapping, a plurality of input mesh vertices that correspond to the plurality of model-generated vertices; and highlight the region of the subject by modifying how the plurality of input mesh vertices appear in a rendering of the input mesh of the subject.

Example 17: The computer-readable storage medium of Example 16, the plurality of model-generated vertices that correspond to the region name are obtained from a predefined region-vertex mapping of parameterized model that maps regions to sets of vertices.

Example 18: The computer-readable storage medium of Example 17, wherein the region name is one of a predefined list of region names of the region-vertex mapping.

Example 19: The computer-readable storage medium of Example 18, wherein the predefined list of region names of the region-vertex mapping includes regions that are joint-segmented.

Example 20: The computer-readable storage medium of Example 17, wherein an additional region of the subject may be identified without training a machine learning model by adding the additional region to the predefined list of region-vertex mappings.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element.

In closing, although the various techniques have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

What is claimed is:

1. A method comprising:
receiving an input mesh of a subject;
identifying a pose of the subject from the input mesh;
generating a parameterized model of the subject from the pose;
receiving a model-generated mesh of the subject from the parameterized model;
generating an input-model vertex mapping from vertices of the model-generated mesh to corresponding vertices of the input mesh;
receiving a region name of a region of the subject;
identifying a plurality of model-generated vertices of the model-generated mesh corresponding to the region name;
identifying, from the input-model vertex mapping, a plurality of input mesh vertices that correspond to the plurality of model-generated vertices; and
highlighting the region of the subject by modifying how the plurality of input mesh vertices appear in a rendering of the input mesh of the subject.

2. The method of claim 1, wherein the representation comprises a real-time 3D representation of the subject.

3. The method of claim 1, wherein the subject comprises a medical patient and the region name is identified by a medial professional.

4. The method of claim 1, wherein the pose is defined by joint positions inferred from the input mesh of the subject.

5. The method of claim 4, wherein the parameterized model represents the subject with a plurality of body parts connected by a plurality of joints, and wherein the parameterized model is parameterized with the joint positions and joint angles of the pose.

6. The method of claim 1, further comprising:
moving one or more vertices of the model-generated mesh of the subject to align with one or more corresponding vertices of the input mesh of the subject.

7. The method of claim 6, wherein the one or more vertices of the model-generated representation are moved independently of each other.

8. The method of claim 7, wherein the one or more vertices of the model-generated representation are moved according to a multi-layer perceptron trained to conform a pair of mesh representations.

9. A system comprising:
a processing unit; and
a non-transitory computer-readable storage medium having computer-executable instructions stored thereupon, which, when executed by the processing unit, cause the processing unit to:
receive an input mesh of a medical patient;
identify a pose of the medical patient from the input mesh;
generate a parameterized model of the medical patient from the pose;
receive a model-generated mesh of the medical patient from the parameterized model;
generate an input-model vertex mapping from vertices of the model-generated mesh to corresponding vertices of the input mesh;
receive a region name of a region of the medical patient from a medical practitioner;
identify a plurality of model-generated vertices of the model-generated mesh corresponding to the region name;
identify, from the input-model vertex mapping, a plurality of input mesh vertices that correspond to the plurality of model-generated vertices; and
highlight the region of the medical patient by modifying how the plurality of input mesh vertices appear in a rendering of the input mesh of the subject.

10. The system of claim 9, wherein the input-model generated vertex mapping associates a vertex of the model-generated mesh at a location of the medical patient with a vertex of the input mesh that is closest to the location of the medical patient.

11. The system of claim 9, wherein the region name is identified by extracting a noun phrase from text or speech produced by the medical practitioner, computing an embedding vector of the noun phrase, and determining that the embedding vector is within a defined distance of an embedding vector representation of the region of the medical patient.

12. The system of claim 11, wherein the computer-executable instructions further cause the processing unit to:
reproduce the text or speech received from the medical practitioner with a local display that is observable by the medical patient.

13. The system of claim 12, wherein the text or speech received from the medical practitioner is processed by a language simplification engine to make the text or speech more understandable to the medical patient.

14. The system of claim 9, wherein the region is highlighted by changing a color of the plurality of input mesh vertices displayed by a local display.

15. The system of claim 9, wherein the computer-executable instructions further cause the processing unit to:
analyze facial expression and body language of the medical patient to detect that the medical patient is confused; and
alert the medical practitioner that the medical patient is confused.

16. A non-transitory computer-readable storage medium having encoded thereon computer-readable instructions that when executed by a processing unit causes a system to:
receive an input mesh of a subject;
identify a pose of the subject from the input mesh;
generate a parameterized model of the subject from the pose;
receive a model-generated mesh of the subject from the parameterized model;
generate an input-model vertex mapping from vertices of the model-generated mesh to corresponding vertices of the input mesh;
receive a region name of a region of the subject;
identify a plurality of model-generated vertices of the model-generated mesh corresponding to the region name;
identify, from the input-model vertex mapping, a plurality of input mesh vertices that correspond to the plurality of model-generated vertices; and
highlight the region of the subject by modifying how the plurality of input mesh vertices appear in a rendering of the input mesh of the subject.

17. The computer-readable storage medium of claim 16, the plurality of model-generated vertices that correspond to the region name are obtained from a predefined region-vertex mapping of parameterized model that maps regions to sets of vertices.

18. The computer-readable storage medium of claim 17, wherein the region name is one of a predefined list of region names of the region-vertex mapping.

19. The computer-readable storage medium of claim 18, wherein the predefined list of region names of the region-vertex mapping includes regions that are joint-segmented.

20. The computer-readable storage medium of claim 17, wherein an additional region of the subject may be identified without training a machine learning model by adding the additional region to the predefined list of region-vertex mappings.
